(19) United States
(12) Patent Application Publication
(10) Pub. No.: US 2019/0015650 A1
(43) Pub. Date: Jan. 17, 2019

(54) MICRONEEDLE TATTOO PATCHES AND USE THEREOF

(71) Applicant: Massachusetts Institute of Technology, Cambridge, MA (US)

(72) Inventors: Ana Jaklenec, Lexington, MA (US); Kevin J. McHugh, Watertown, MA (US); Robert S. Langer, Newton, MA (US)

(21) Appl. No.: 16/036,712

(22) Filed: Jul. 16, 2018

Related U.S. Application Data
(60) Provisional application No. 62/533,081, filed on Jul. 16, 2017.

(51) Int. Cl. A61M 37/00; A61B 5/00; A61B 90/90; A61B 50/30; A61K 9/00; A61K 49/00; A61K 8/02; A61Q 1/02; A61K 49/18

(52) U.S. Cl. CPC A61M 37/0015; A61B 2090/3941; A61B 5/0071; A61B 90/90; A61B 50/30; A61K 9/0021; A61K 49/0069; A61K 8/0216; A61Q 1/02; A61K 49/18; A61M 2037/0023; A61M 2037/0046; A61M 2037/0061; A61M 2202/30; A61M 37/0076

(57) ABSTRACT

Microneedle patches have been developed that can be used to deliver therapeutic, prophylactic, diagnostic agents and/or dyes to the skin. The microneedles encapsulate the agent(s) to be delivered. These are formed of a biodegradable polymer that dissolves upon insertion into skin or tissue, so that the microneedles break off from the substrate forming the patch, remaining in the skin/tissue at the site of insertion. The patches are used to create a tattoo or to deliver therapeutic, prophylactic or diagnostic agent in combination with a tattoo. In one embodiment, the microneedle patch contains both vaccine and dye pigments to administer vaccine and record such administration in one application of the microneedle patch.

### CROSS-REFERENCE TO RELATED APPLICATIONS

This application claims priority to U.S. Ser. No. 62/533,081, filed on Jul. 16, 2017, which is incorporated herein in its entirety.

### STATEMENT REGARDING FEDERALLY SPONSORED RESEARCH

None.

### FIELD OF THE INVENTION

The invention relates generally to disposable one-time use microneedle tattoo patches, which may have applications in creating records simultaneously with drug delivery, to make tattoos not visible to the eye, and in agricultural applications.

### BACKGROUND OF THE INVENTION

Tattoos are generally divided into two groups—permanent and temporary. People have tattooed patterns and symbols on their skin for thousands of years, typically using a sharp object to disrupt the skin surface, and then rubbing into the wound dyes, pigments, and charcoal. These remain trapped in the skin as it heals.

In agriculture, permanent tattoos and brands (burn scars) have been used to indicate ownership. In the U.S., regulatory agencies require animals to be individually marked to share origin, to help control disease. These may be in the form of tattoos, typically made by clamping needle letters and numbers, into the inside of the ear, or more recently, using RFID tags or microchip implants. The latter are expensive, however, and may migrate. In people, elaborate tattoo machines have been developed to create colorful, detailed designs, using a mechanized needle connected to one or more dye reservoirs.

There are a number of temporary tattoos. One of the oldest was the application of ocher to the skin, more recently patterns created by plant dyes such as henna. Currently tattoos can be applied to the skin using temporary decorative skin decals that wear away in relatively short amounts of time, typically between hours and weeks. Temporary tattoo market relies on the tattoo be either on an image printed on a skin adherent material, or skin stain. For example, one type of sticker-based tattoo contains a printed image on a release sheet that is placed on a backing sheet, where the image is transferred to the skin when the backing sheet is removed. This leaves tattoo patterns on the skin that wear off in a little over a week. Airbrush tattoo is another type of temporary tattoos that is created by spraying dye pigment over a tattoo stencil placed over the skin. The dye pigment stain lasts for couple of months.

There is no currently available means of applying a permanent tattoo that is not invasive and painful. There is no currently available device for making a permanent tattoo that is disposable, individualizable, and relatively painless and non-invasive. There is no currently available device to apply a therapeutic, prophylactic or diagnostic agent in combination with a tattoo to identify the agent, the date, and/or the individual to whom it is administered. There is no device that one can use to form a tattoo which is invisible in regular light.

Therefore, it is an objective of the present invention to provide such a device.

It is another objective of the present to provide method of making and using such a device to allow painless, facile, and quick application of the dyes to the skin.

### SUMMARY OF THE INVENTION

Microneedle patches have been developed. These can be used to deliver therapeutic, prophylactic, diagnostic and/or dyes (including dyes, pigments, fluorophores, etc., collectively referred to herein as “dyes”) agents to the skin. The microneedles encapsulate the agent(s) to be delivered. These are formed of a biodegradable polymer that dissolves upon insertion into skin or tissue, so that the microneedles break off from the substrate forming the patch, remaining in the skin/tissue at the site of insertion. The polymer continues to degrade, leaving the agent(s) at the site of insertion.

In a preferred embodiment, the patches are used to create a tattoo. In another, the patch is used to deliver therapeutic, prophylactic or diagnostic agent in combination with a tattoo. In one embodiment, the tattoo is invisible in normal light, being visible in the infrared, fluorescent or ultraviolet light. The diameter and length of the microneedles, the agent to be imaged, and the particle size and location in the microneedles, as well as the composition, are selected to be compatible with the agent to be delivered, as well as to deliver a sufficient amount of agent at the desired site to be effective, to minimize pain, and to release from the patch in a desired time frame, preferably five minutes or less.

Active agents may be encapsulated in the microneedles for delivery through the skin of a subject. In one embodiment, vaccine is delivered through the microneedle patch. In another embodiment, the microneedle patch contains both vaccine and dye pigments to administer vaccine and record such administration in one application of the microneedle patch.

Exemplary dyes include inorganic nanocrystals, lanthanide-based dyes, other fluorophores, and non-fluorescent imaging agents. Preferably the dye is a near infrared imaging agent with an excitation wavelength and an emission wavelength in the near infrared range. A preferred type of inorganic nanocrystals is quantum dots, e.g., copper-based quantum dots or silver-based quantum dots.

Dyes are generally encapsulated in polymeric particles prior to embedding in the microneedle structure. Particles protect or diminish the photobleaching of an encapsulated dye, providing a protective environment for increasing the photostability of dyes against changes in the pH or an oxidative environment. In preferred embodiments, slow degrading microparticles are used to encapsulate dyes at a high loading efficiency with minimal leakage.

The arrangement of microneedles (size, spacing distance, quantity, density, etc.) as well as the type of dyes therein, may correspond to unique information such as a vaccination record, date, or identification of a subject. The microneedles dissolve or are degraded within 3, 4, 5, 6, 7, 8, 9, 10, or 15 minutes upon contact with skin, delivering the dye-encapsulated particles in the skin (preferably the dermis), leaving the dyes as markings/tattoos that last at least five years. These tattoos are especially useful as medical decals as a “on-patient” record of medical history: e.g., sub dermal immunization record (individual vaccination history), blood type or allergens.

A microneedle pattern, a combination of imaging dyes, or both may be used to encode multiple pieces of information in one microneedle patch. The concept is to use this to aid healthcare workers who have to act on very little patient information. Ideally the marking would not be visible to the naked eye but could be visualized using a device as simple as a cell phone from which the it or uv filters have been removed.

The patches have many advantages. They are easily mass produced, stored and shipped. They are easily applied without conventional needles and relatively painless. No bio-hazardous sharps are generated through the application of biodegradable microneedles.

The patches have applications in the defense industry, as a well to mark soldiers without using invasive means such as a chip, or means such as a “dog tag” which may be lost, providing an alternative means of identification or medical record, optionally while at the same time administering vaccines.

The patches may also be used to apply dyes for cosmetic purposes, such as lip enhancement, eyebrow darkening, or delivery of an agent such as botulinum toxin or growth factor to alleviate wrinkles.

The patches also have applications in the animal industry, providing a clean, relatively easy and painless way to permanently identify animals. The patches can be made so that the marking include a group identify (such as the USDA farm identification number) as well as individual identify.

The microneedles can be prepared by first creating a master mold using a material such as poly dimethyl siloxane (PDMS), based on the geometries created with CAD; followed by solidifying the solution/suspension containing biodegradable materials along with dye (fluorescent/non-fluorescent) or particles encapsulating dyes, therapeutic, prophylactic or diagnostic agent.

### BRIEF DESCRIPTION OF THE DRAWINGS

FIGS. 1A and 1B are schematics showing the workflow of tattoo implantation in the skin and an imaging process with dye (FIG. 1A) or fluorophore (FIG. 1B).

FIGS. 2A-2C are line graphs showing the absorbance spectra of IRDC3 (FIG. 2A), copper quantum dots (FIG. 2B), and silver quantum dots (FIG. 2C), respectively, with the absorbance spectra of melanin in the background.

FIGS. 3A-3C are line graphs showing the emission spectra of IRDC3 (FIG. 3A), copper quantum dots (FIG. 3B), and silver quantum dots (FIG. 3C), respectively, with the absorbance spectra of melanin in the background.

FIGS. 4A-4C are dot graphs showing the percentage of remaining fluorescence intensity of IRDC3 (FIG. 4A), silver quantum dots encapsulated in poly(methyl methacrylate) particles (FIG. 4B), and copper quantum dots encapsulated in poly(methyl methacrylate) particles (FIG. 4C), respectively, over days of photobleaching ex vivo.

FIG. 5 is a spectra of absorbance over wavelength (nm) for water, Hb, HbO₂, and melanin.

FIG. 6 is a line graph showing the signal-to-noise ratios of lanthanide dye, IRDC2 when excited at 635 nm.

FIG. 7 is a line graph showing the signal-to-noise ratios of lanthanide dye, IRDC3 when excited at 808 nm.

FIG. 8A shows a schematic depicting the potential reduction of quantum yields of dyes due to absorbance of wavelengths by melanin and/or deeper tissue. When an excitation light shines on the skin, it may be absorbed by melanin and/or the deeper tissue before reaching the fluorophore. The excited fluorophore emits at a wavelength that may be absorbed by the tissue and/or melanin before emitting off the skin.

FIGS. 8B and 8C are graphs of the intensity per gram of dye (8B) and intensity per gram of particles (8C).

FIGS. 9A-9C are line graphs showing the percent of fluorescent intensities over time (minutes) of dyes that were exposed to light from a compact fluorescent (CFL) bulb (FIG. 9A), were submerged in 3 micromolar hydrogen peroxide (FIG. 9B), and were submerged in a pH5 environment (FIG. 9C), respectively.

FIG. 10 is a cross-sectional schematic of the polymeric particles containing imaging agents.

FIG. 11 is a graph of intensity versus filter wavelength (nm).

FIG. 12A shows the optimal microneedle shape and dimensions.

FIGS. 12B and 12C are graphs showing optimal microneedle dimensions for pig ear (12B) and SynDerm (12C).

### DETAILED DESCRIPTION OF THE INVENTION

Unlike decorative tattoos, markings on the skin to encode medical history or medical information is challenging primarily due to the lack of appropriate inks or dyes for years long photostability and the device to administer or image them off the skin. There is no existing technology in the market that will store medical history with the aid of microneedle-based tattoo, although radio frequency identification (RFID) technology based implantable electronic chips are used under the skin.

Topical delivery of therapeutic active agents (or imaging agents) is a very useful method for achieving systemic or localized pharmacological effects. The main challenge in transcutaneous drug delivery is providing sufficient drug penetration across the skin. The skin consists of multiple layers starting with a stratum corneum layer about (for humans) 20 microns in thickness (comprising dead cells), a viable epidermal tissue layer about 70 microns in thickness, and a dermal tissue layer about two mm in thickness.

Current topical drug delivery methods are generally based upon the use of penetration enhancing methods, which often cause skin irritation, and the use of occlusive patches that hydrate the stratum corneum to reduce its barrier properties. Allowing large fractions of topically applied drug to penetrate through skin is still highly challenging with very poor efficiency.

I. Reagents and Device

A. Microneedle Patch

1. Biodegradable Microneedles

Methods of making microneedles are well known. These are typically formed using casting into a mold, but may also be created using other available methods.

The material forming the microneedles is critical. It must be biodegradable and it must degrade sufficiently within a few minutes of insertion into the skin for the microneedle to break loose from the substrate and stay at the site of administration. It must then continue to degrade to release the agent and/or dye at the site of administration. In the preferred embodiment, the patch is pressed upon the skin for five minutes and the agent and/or dye deposited sub-dermally upon the dissolution of the microneedles.

In one embodiment, microneedles are fabricated from a combination of polyvinyl alcohol (PVA) and polyvinyl pyrrolidone (PVP). In another embodiment, microneedles are fabricated from a sugar-based material such that they are dissolvable at the site of administration.

Alternative materials for forming the degradable portion of the microneedles include hydroxy acids such as lactic acid and glycolic acid polyglycolide, polylactide-co-glycolide, and copolymers with PEG, polyanhydrides, poly(ortho)esters, polyurethanes, poly(butyric acid), poly(valeric acid), and poly(lactide-co-caprolactone). Most of these need to include additives to increase the rate of dissolution upon administration.

Optionally, the microneedle may contain other materials, including metals, ceramics, semiconductors, organics, polymers, and composites. Preferred materials of construction include pharmaceutical grade stainless steel, gold, titanium, nickel, iron, gold, tin, chromium, copper, alloys of these or other metals, silicon, silicon dioxide, and polymers.

The type of biodegradable materials (e.g., polymers) to form microneedle and/or their concentration(s) in forming microneedle are selected to provide sufficient dissolution rates in vivo or upon contacting the skin. Exemplary dissolution rates include within 1, 2, 3, 4, 5, 6, 7, 8, 9, 10, or 15 minutes of application to the skin, at least the tip of microneedles or the portion having embedded therein dyes or dyes encapsulated in microparticles dissolves in the skin such that the embedded dyes or microparticles encapsulating the dyes are released or deposited into the skin.

Microneedles typically penetrate deep into the dermis to prevent the dye-containing particles from shedding with skin. For example, microneedles may have a cylindrical body of a height between 0.5 mm and 6 mm, preferably between 1 mm and 4 mm, more preferably between 1.5 mm and 2 mm. Microneedles may have a tip that is conical shaped or beveled, where the tip is of a height or length between 0.1 mm and 1.2 mm, preferably between 0.2 mm and 0.8 mm, more preferably between 0.3 mm and 0.4 mm. A lower insertion force is needed for applying sharp microneedles. These geometries allow sharpness (radius of curvature) of the microneedles that are superior to traditional microneedles that are 19 G or 25 G.

In one embodiment, microneedles have a height of 1,500 μm and a base of 300 μm thick.

The microneedles may be arranged into an array of m×n microneedles within an area (e.g., 1 cm², 10 cm², or 50 cm²) where m and n are independently integers between 2 and 100 or greater. Laser cutting may guide the distribution of the microneedles. The array may outline a square, rectangle, diamond, or round shape. The spacing or the smallest distance between two adjacent microneedles in an array may be the same for any two microneedles, or may be different resulting in an array with a denser section of microneedles and a less dense section.

The microneedles are generally edged, preferably a substantially sharp edge to assist in penetrating the stratum corneum and epidermis and into the dermis. The edged microneedles generally have a tip that is a conical shape or beveled.

2. Patch Substrate

The patches consist of a flexible substrate having microneedles formed thereon, the microneedles containing therapeutic, prophylactic, or diagnostic agent and/or dyes encapsulated or dispersed therein, preferably first encapsulated in microparticles.

The substrate, or base element, includes a substrate to which the microneedles are attached or integrally formed. The base element may be a patch with elongated microneedles. The patch may be formed from the same material as that for the microneedles, or different. The base element can be constructed from a variety of materials, including metals, ceramics, semiconductors, organics, polymers, and composites. The base element is generally thick enough for maneuvering; or it may be thin enough to be a sticky film for application on the skin to remain contact with the skin during the period in which the degradable microneedles dissolve in the dermis to release the dyes or particles encapsulating the dyes.

The microneedles can be oriented perpendicular or at an angle to the base element. Preferably, the microneedles are oriented perpendicular to the substrate so that a larger density of microneedles per unit area of substrate can be provided. An array of microneedles can include a mixture of microneedle orientations, heights, or other parameters.

In a preferred embodiment of the device, the base element and/or microneedles, as well as other components, are formed from flexible materials to allow the device to fit the contours of the biological barrier, such as the skin, to which the device is applied. A flexible device will facilitate more consistent penetration during use, since penetration can be limited by deviations in the attachment surface. For example, the surface of human skin is not flat due to dermatoglyphics (i.e. tiny wrinkles) and hair.

In some embodiments, the microneedle array is constructed in the form of a microneedle “patch” that is attached to the skin at the time the dye is to be transferred from the microneedles to the skin (preferably the dermis).

3. Agents to be Encapsulated in Microneedles

There are two categories of agents to be delivered: therapeutic, prophylactic and diagnostic agents (referred to herein as “agents”) and dyes, pigments, metals, fluorophores, inks (referred to herein as “dyes”).

a. Dyes

A dye for marking the skin is prepared from a material that may transmit through pigmented skin, be resistant to photobleaching, be safe to the subject to which the microneedle is applied, have a relatively high quantum yield, be amenable to be loaded in particles at a high loading amount, have a low background noise, and/or be stable to variations in temperature, pH, or oxidation in the in vivo environment, for at least one year, 2 years, 3 years, 4 years, 5 years, or longer.

In some embodiments, as shown in FIG. 10, the dyes are encapsulated in polymeric particles such as poly(methyl methacrylate) (PMMA) particles or polystyrene particles, which improves the safety profile, for example, resulting in reduced toxicity compared to delivering the dye directly in the microneedles without the PMMA particles, measurable by lowered level of apoptosis of cells by at least 10%, 20%, 30%, 40%, 50%, 60%, 70%, 80%, or 90% following application of the microneedles in the skin.

Signal-to-noise (S/N) ratio in imaging an imaging agent from within the skin may be generally described by the formula:

S/N=[(1−Tissue Absorbance)×Particle Loading×Quantum Yield×(1−Photobleaching and environmental degradation rate)]÷Background noise.

Preferably the dyes for marking the skin have a S/N ratio of at least about 5, preferably at least about 15, and may be between about 50 and 150.

Preferably the marking would not be visible to the naked eye.

Inorganic Nanocrystals

Semi-conducting nanocrystals have customizable wavelengths have high quantum yields. An exemplary semi-conducting nanocrystal is near infra-red (NIR) emitting, fluorescent inorganic crystal. NIR emitting crystals emit in the range between about 900 nm and about 1,000 nm and the fluorescence is to the naked eye. These inorganic crystals provide markings under the skin, where the markings are invisible to the naked eye and may be illuminated for visualization with appropriate imaging device.

In some embodiments, the NIR emitting inorganic dye is semiconducting nanocrystals of copper or silver, which may be encapsulated in a poly methyl methacrylate (PMMA) microparticle for embedding in the microneedles.

In some embodiments, the dye is a semi-permanent or permanent, in which the dye pigment under skin has a strong photostability. For example, the dye pigment is not degraded or is only degraded for less than 50%, 40%, or 30% under skin after exposure to ambient sun light and ambient environment over the course of 6 months, 1 year, 2 years, 3 years, 5 years, or 10 years or longer. Photostability of a pigment is generally evaluated using high solar irradiance (7× intensity of sea level sun light) after the dye pigment is deposited under melanin pigmented human cadaver skin.

Quantum Dots

One embodiment of a suitable fluorophore is a quantum dot. Quantum dots are very small semiconductor particles, generally only several nanometres in size, so small that their optical and electronic properties differ from those of larger particles. Generally, larger quantum dots (radius of 5-6 nm, for example) emit longer wavelengths resulting in emission colors such as orange or red. Smaller quantum dots (radius of 2-3 nm, for example) emit shorter wavelengths resulting in colors like blue and green, although the specific colors and sizes vary depending on the exact composition of the QD.

Quantum dots are suitable for use as the dye in the microneedles due to their customizable wavelengths, low tissue absorption, high quantum yields, and less toxicity than lanthanide-containing dyes. In some embodiments, the quantum dots are surface modified (or stabilized) with hydrophobic organic ligands to increase hydrophobicity, thus compatibility with certain hydrophobic polymers for high loading amount in polymeric particles shown in FIG. 10. In some embodiments, quantum dots that are cadmium free mitigate potential toxicity to the skin.

Quantum dots as dyes for the microneedles can be produced from an inorganic material, generally inorganic conductive or semiconductive material including group II-VI, group III-V, group IV-VI and group IV semiconductors. Suitable semiconductor materials include, but are not limited to, Si, Ge, Sn, Se, Te, B, C (including diamond), P, BN, BP, BAs, AIN, AlP, AlAs, AlSb, GaN, GaP, GaAs, GaSb, InN, InP, InAs, InSb, AIN, AlP, AlAs, AlSb, GaN, GaP, GaAs, GaSb, ZnO, ZnS, ZnSe, ZnTe, CdS, CdSe, CdSeZn, CdTe, HgS, HgSe, HgTe, BeS, BeSe, BeTe, MgS, MgSe, GeS, GeSe, GeTe, SnS, SnSe, SnTe, PbO, PbS, PbSe, PbTe, CuF, CuCl, CuBr, CuI, Si₃N₄, Ge₃N₄, Al₂O₃, (Al, Ga, In)₂ (S, Se, Te)₃, Al₂CO, and appropriate combinations of two or more such semiconductors.

Synthesis of Dyes

Quantum dots or inorganic nanostructures as dyes for inclusion in microneedles are generally described in U.S. Pat. No. 6,225,198, U.S. Patent Application Publication No. 2002/0066401, U.S. Pat. No. 6,207,229, U.S. Pat. No. 6,322,901, U.S. Pat. No. 6,949,206, U.S. Pat. No. 7,572,393, U.S. Pat. No. 7,267,865, U.S. Pat. No. 7,374,807, U.S. patent application 20080118755, and U.S. Pat. No. 6,861,155.

Exemplary quantum dots for inclusion in the microneedle include low toxicity, high quantum-yield copper-based quantum dots such as copper-indium-selenide with an overlay/film of zinc sulfide (ZnS), optionally doped with aluminum, i.e., CuInSe2/ZnS:Al; as well as silver-based quantum dots such as near-infrared emissive quantum dots having a core of silver-indium-selenide and a shell of ZnS, optionally doped with aluminum, i.e., AgInSe2/ZnS:Al.

Other Fluorophores

Another type of dye suitable for marking in the skin is fluorophores. A fluorophore is a fluorescent chemical compound that can re-emit light upon light excitation. Preferably, fluorophores that are not visible to the naked eye under ambient sun exposure are used as the dye for the microneedles.

In some embodiments, lanthanide-based dyes, IRDC3 or IRDC2, are used as the dye for inclusion in the microneedles.

Non-Fluorescent Dyes

Other exemplary dyes for inclusion in microneedle include non-fluorescent molecules such as paramagnetic molecules, magnetic molecules, and radionuclides.

Tattoo Inks and Dyes

Carbon (soot or ash) is often used for black. Other elements used as pigments include antimony, arsenic, beryllium, calcium, copper, lithium, selenium, and sulphur. Tattoo ink manufacturers typically blend the heavy metal pigments and/or use lightening agents (such as lead or titanium) to reduce production costs. Some pigments include inorganic materials such as ocher.

Natural materials such as henna may also be used.

b. Active Agents

The microneedles are also suitable for delivery of active agents (e.g., therapeutic, prophylactic or diagnostic agents) in addition to or separately from the delivery of the dyes or ink molecules.

In some embodiments, the active agents are encapsulated in, absorbed in, covalently bonded to, or modified onto the surface of, the same microparticles encapsulating the dyes. In other embodiments, the active agents are encapsulated in, absorbed in, covalently bonded to, or modified onto the surface of different particles from those delivering the dyes or ink molecules.

In some embodiments, the active agents are encapsulated in, absorbed in, covalently bonded to the microneedle, which upon the dissolution of the microneedle release into the skin.

Exemplary active agents can be proteins or peptides, sugars or polysaccharides, lipids, nucleotide molecules, or combinations thereof, or synthetic organic and inorganic compounds such as a low molecular weight compound having a molecular weight of less than 2000 D, more preferable less than 1000 D.

A preferred active agent is a vaccine antigen. Other agents include insulin, anti-infectives, hormones, growth regulators, and drugs for pain control. Typically the agent is administered in a dosage effective for local treatment.

The microneedle array is also useful for delivering specific compounds or actives into the skin, such as cosmetic compounds or nutrients, or various skin structure modifiers that can be delivered subcutaneously without having to visit a cosmetic surgery clinic. In addition, color cosmetics could also be delivered subcutaneously to provide long-term benefits for the skin, and even makeup or lipstick-type coloring compounds can be delivered by use of the microneedle patches. The color cosmetics are delivered into the epidermis or the dermis, where they remain in place for at least one or two months, or even longer (e.g., years). Since the epidermis is renewable, agents that are delivered there would eventually wear out; and then will be expunged from the body. This allows a person to change their “look” according to changes in fashion and style, which typically change every season.

4. Microparticles for Encapsulation of the Dyes and/or Active Agents

In preferred embodiments, microparticles are used to encapsulate the dye and/or agent and provide an environment in which the dye and/or agent is chemically stabilized or provided with physical protection, e.g., reduced or minimal photobleaching or other negative impact in the biological environment.

In certain embodiments, the microparticles are slow degrading particles such that encapsulated dyes are protected for 1 month, 2 months, 3 months, 6 months, 1 year, 2 years, 5 years or greater.

In some embodiments, the microparticles may reduce the oxidation of encapsulated dyes by at least 50%, 60%, 70%, 80%, 90%, or more. For example, encapsulation of IRDC3 in particles reduces the oxidizing effect of 3 micromolar hydrogen peroxide by 98%.

In some embodiments, microparticles are also used to shield the skin from toxicity associated with the dye or with high concentration of the dye. Microparticles generally do not interfere with the illumination or the emission or the dye signal through the skin.

Microparticles or nanoparticles for encapsulating dyes are generally prepared with bio-inert materials. The size of microparticles is selected to allow a high loading of the dye or the active agents and to support long residence time in the skin.

Exemplary polymers include, but are not limited to, polymers prepared from lactones such as poly(caprolactone) (PCL), polyhydroxy acids and copolymers thereof such as poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), and blends thereof, polyalkyl cyanoacralate, polyurethanes, polyamino acids such as poly-L-lysine (PLL), poly(valeric acid), and poly-L-glutamic acid, hydroxypropyl methacrylate (HPMA), polyanhydrides, polyorthoesters, poly(ester amides), polyamides, poly(ester ethers), polycarbonates, ethylene vinyl acetate polymer (EVA), polyvinyl alcohols (PVA), polyvinyl ethers, polyvinyl esters such as polyvinyl acetate), polyvinyl halides such as poly(vinyl chloride) (PVC), polyvinylpyrrolidone, polysiloxanes, polystyrene (PS), celluloses including derivatized celluloses such as alkyl celluloses, hydroxyalkyl celluloses, cellulose ethers, cellulose esters, nitro celluloses, hydroxypropylcellulose, and carboxymethylcellulose, polymers of acrylic acids, such aspoly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobut 1 acrylate), poly(octadecyl acrylate) (jointly referred to herein as “poly aery lie acids”), polydioxanone and its copolymers, polyhydroxyalkanoates, polypropylene fumarate, polyoxymethylene, poloxamers, poly (butyric acid), trimethylene carbonate, and polyphosphazenes.

B. Imaging

The tattoos may be visible or may be “hidden” so that they are visualized only ben exposure to IR or UV or other special lights.

The tattoos may be used to create any image and/or for identification or unique signature

Arrays of microneedle may be designed to indicate the identification of specific vaccination or other specific medical information. For example, the number of microneedles, their organization/orientation, their spacing distance, and/or the specific type of dye(s) incorporated in the microneedles may individually or in combination correlate to a specific information to be stored under skin, i.e., a signature.

The type of dyes may be selected to indicate the identification of specific vaccination or other specific medical information. For example, dyes or ink molecules having different excitation/illumination wavelengths and/or having different emission wavelengths may be applied through different microneedles to correspond to different vaccinations, medicine administrations, or other medical procedures.

Patches containing microneedles can be actuated manually with a human finger, or electrically using an electrochemical gas generator.

For imaging of the dye or tattoo on the skin, a device is used to illuminate or visualize, and optionally captures and stores, the information of illuminated dye or tattoo. For example, a portable device or a cellular phone with some imaging capabilities may be modified to visualize the marking on the skin.

Standard devices may be used, or modified to include a source for excitation, an emission filter, a power supply (e.g., battery), and/or integration with the case of a device, as well as an appropriate user interface for initiating the imaging, storing the information from the markings, and/or identifying the information from the markings.

For example, a cell phone can be modified for visualization of images not visible under standard light. Generally a laser diode and batter are integrated into a phone case to produce light with the correct excitation wavelength for a dye. For imaging an NIR dye, the stock IR filter on the phone camera is removed; and a long- or band-pass filter is added on top of the camera lens to filter out unwanted light.

In one embodiment, a smart phone (e.g., GOOGLE, NEXUS) can be modified by adding an external low powered NIR laser diode (808 nm) and an adjustable collimator. In one embodiment, a band pass filter is placed over camera piece so that camera only registers emission wavelengths from 900-1000 nm, suitable for imaging NIR emitting, inorganic nanocrystals. In a preferred embodiment, the phone is modified to use a 780 nm LED with an 800 nm short-pass filter. In another embodiment, an 850 nm long-pass color glass filer was used in series with the dielectric filter to reduce background signal. Dielectric filters are generally sharper and have a more complete cutoff. The two filters reduce the increased background signal. For imaging NIR emitting, inorganic nanocrystals, the IR cut off filter was removed from the smart phone camera module. An external circuit that powers the laser diode has a power button so that laser can be powered on from the outside.

Suitable software is typically installed in the device (e.g., cellular phone) to process the detected images and identify the markings onboard the phone to eliminate potential user error. The software may include grayscaling, binarization, and noise reduction algorithms to optimize the signal for detection. In some embodiments of processing images of IRDC3, a near infrared dye, the software generates a square around the detected fluorophores.

II. Method of Preparation

A. Fabrication

1. Fabrication of Microneedles

Microneedles typically are long enough and sharp enough to penetrate deep into the dermis. These long and sharp microneedles may be difficult to achieve using traditional microfabrication techniques. A different fabrication process is used involving a mold.

First the geometries of microneedles are created in a computer-assisted drawing (CAD) software. Microneedle master mold can be prepared from two-photon polymerization, based on the geometries created with CAdD, and the fabricated needle design is transferred to a poly dimethyl siloxane (PDMS) solution, which hardens to form a complementary mold of the needles. A solution of the biodegradable solution mix along with dye (fluorescent/non-fluorescent) pigment is added to the PDMS mold, centrifuged and vacuumed for a sufficient time (e.g., overnight) to remove any trapped air bubbles. The resulting microneedle patch is peeled from the PDMS mold.

Alternatively, an array of microneedles are manufactured by a micromolding method, a microembossing method, or a microinjection method. For example, microfabrication processes that may be used in making the microneedles include lithography; etching techniques, such as wet chemical, dry, and photoresist removal; thermal oxidation of silicon; electroplating and electroless plating; diffusion processes, such as boron, phosphorus, arsenic, and antimony diffusion; ion implantation; film deposition, such as evaporation (filament, electron beam, flash, and shadowing and step coverage), sputtering, chemical vapor deposition (CVD), epitaxy (vapor phase, liquid phase, and molecular beam), electroplating, screen printing, lamination, stereolithography, laser machining, and laser ablation (including projection ablation). See generally Jaeger, Introduction to Microelectronic Fabrication (Addison-Wesley Publishing Co., Reading Mass. 1988); Runyan, et al., Semiconductor Integrated Circuit Processing Technology (Addison-Wesley Publishing Co., Reading Mass. 1990); Proceedings of the IEEE Micro Electro Mechanical Systems Conference 1987-1998; Rai-Choudhury, ed., Handbook of Microlithography, Micromachining & Microfabrication (SPIE Optical Engineering Press, Bellingham, Wash. 1997).

2. Encapsulation of dyes or agents in particles

Dyes or agents may be encapsulated in particles via one or more techniques to allow a high loading amount between about 5% and 80% (wt/wt), between about 10% and 50% (wt/wt), or about 10%, 20%, 30%, 40%, or 50% wt/wt.

Therapeutic, prophylactic or diagnostic agents may be encapsulated in the same microparticles encapsulating the dyes or in different particles. Such particles encapsulating the therapeutic or prophylactic agents are capable of controlled release of the therapeutic or prophylactic agents into the skin.

Suitable techniques for making polymeric particles for encapsulation of dyes and agents include, but are not limited to, emulsion, solvent evaporation, solvent removal, spray drying, phase inversion, low temperature casting, and nanoprecipitation. The imaging agent, the therapeutic or prophylactic agents, and pharmaceutically acceptable excipients can be incorporated into the particles during particle formation.

In one embodiment, NIR dyes are milled to hundreds of nanometers before encapsulation. They may be encapsulated in PMMA particles using a double-emulsion technique. In some embodiments, the particles are prepared with non-degradable materials to encapsulate a dye in order to assay a separate release-based (e.g., leaching of dyes from particles) loss in signal from other factors such as photo-bleaching.

Emulsion or Solvent Evaporation

In this method, the polymer(s) are dissolved in a volatile organic solvent, such as methylene chloride. The organic solution containing the polymer is then suspended in an aqueous solution that contains an emulsifier, e.g., a surfactant agent such as poly(vinyl alcohol) typically under probe sonication for a period of time (e.g., 2 minutes) to form an emulsion. The dyes and/or active agents may be dissolved in the organic solvent with the polymer or in the aqueous solution, depending on its hydrophilicity/hydrophobicity. The emulsion is added to another large volume of the emulsifier with magnetic stirring to evaporate the organic solvent. The resulting emulsion is stirred until most of the organic solvent evaporated, leaving solid nanoparticles. The resulting particles are washed with water and dried overnight in a lyophilizer. Particles with different sizes and morphologies can be obtained by this method.

Solvent Removal

In this method, the polymer, the dyes and/or active agents, and other components of the particles are dispersed or dissolved in a suitable solvent. This mixture is then suspended by stirring in an organic oil (such as silicon oil) to form an emulsion. Solid particles form from the emulsion, which can subsequently be isolated from the supernatant.

Spray Drying

In this method, the polymer, the dyes and/or the active agents, and other components of the particles are dispersed or dissolved in a suitable solvent. The solution is pumped through a micronizing nozzle driven by a flow of compressed gas, and the resulting aerosol is suspended in a heated cyclone of air, allowing the solvent to evaporate from the microdroplets, forming particles.

Phase Inversion

In this method, the polymer, the dyes and/or the active agents, and other components of the particles are dispersed or dissolved in a “good” solvent, and the solution is poured into a strong non solvent for the polymeric components to spontaneously produce, under favorable conditions, nanoparticles or microparticles.

Low Temperature Casting

Methods for very low temperature casting of particles are described in U.S. Pat. No. 5,019,400 to Gombotz et al. In this method, the polymer the dyes and/or the active agents, and other components of the particles are dispersed or dissolved is a solvent. The mixture is then atomized into a vessel containing a liquid non-solvent at a temperature below the freezing point of the solution which freezes the polymer, the dyes and/or the active agents, and other components of the particles carrier as tiny droplets. As the droplets and non-solvent for the components are warmed, the solvent in the droplets thaws and is extracted into the non-solvent, hardening the particles.

3. Prepare Microneedles with Embedded Particles Encapsulating Dyes and/or Active Agents

Particles encapsulating dyes and/or active agents may be blended or mixed with the polymer solution/suspension in a mold in forming the solidified microneedles with such particles embedded therein.

B. Sterilization and Packaging

The microneedles and substrate or base element to which the microneedles are attached to or integrally formed are generally sterilized and packaged for storage and shipping. Formed microneedles and the base element may be sterilized via gamma irradiation, UV sterilization, or other techniques that do not interfere or damage the physical structure and the electro-optical properties of encapsulated dyes.

III. Methods of Use

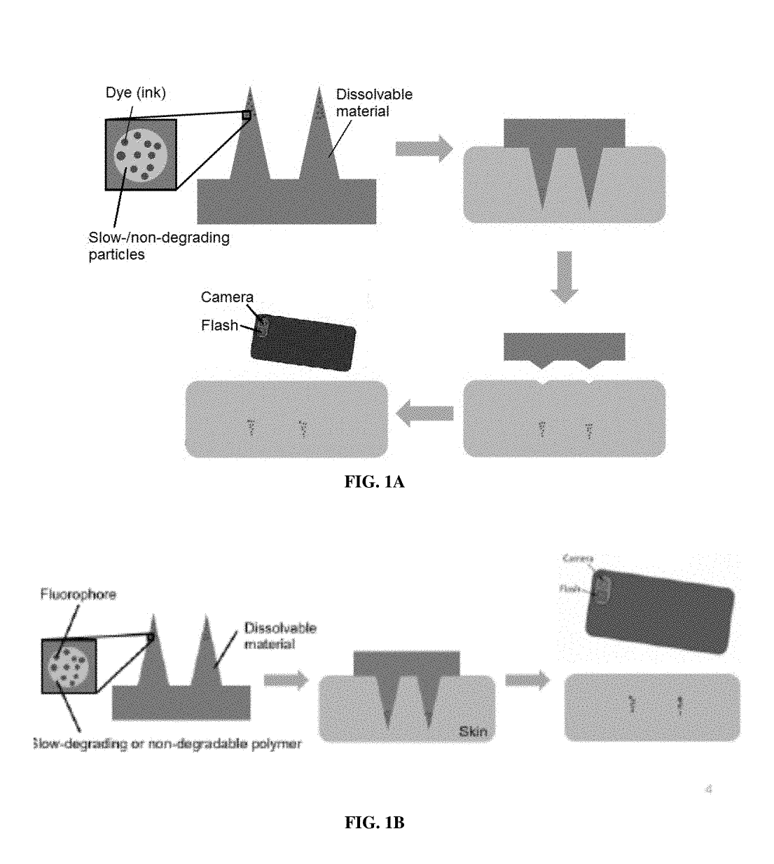

FIGS. 1A and 1B are schematics showing the workflow of tattoo implantation in the skin and an imaging process with dye (FIG. 1A) or fluorophore (FIG. 1B).

The arrangement of microneedles (size, spacing distance, quantity, density, etc.) as well as the type of dyes therein, may correspond to unique information such as a vaccination record, date, or identification of a subject. The microneedles dissolve or are degraded within 3, 4, 5, 6, 7, 8, 9, 10, or 15 minutes upon contact with skin, delivering the dye-encapsulated particles in the skin (preferably the dermis), leaving the dyes as markings/tattoos that last at least five years. These tattoos are especially useful as medical decals as an “on-patient” record of medical history: e.g., sub dermal immunization record (individual vaccination history), blood type or allergens.

A microneedle pattern, a combination of imaging dyes, or both may be used to encode multiple pieces of information in one microneedle patch. The concept is to use this to aid healthcare workers who have to act on very little patient information. Ideally the marking would not be visible to the naked eye but could be visualized using a device as simple as a cell phone from which the it or uv filters have been removed.

The patches have many advantages. They are easily mass produced, stored and shipped. They are easily applied without conventional needles and relatively painless. No bio-hazardous sharps are generated through the application of biodegradable microneedles.

The patches have applications in the defense industry, as a well to mark soldiers without using invasive means such as a chip, or means such as a “dog tag” which may be lost, providing an alternative means of identification or medical record, optionally while at the same time administering vaccines.

The patches may also be used to apply dyes for cosmetic purposes, such as lip enhancement, eyebrow darkening, or delivery of an agent such as botulinum toxin or growth factor to alleviate wrinkles. An advantage of the patch is that it can be trimmed or shaped just before use to personalize the tattoo to the individual and site of application.

The patches also have applications in the animal industry, providing a clean, relatively easy and painless way to permanently identify animals. The patches can be made so that the marking include a group identify (such as the USDA farm identification number) as well as individual identify.

In one embodiment, the microneedle patch is used to generate a sub-dermal marking system that can be used to track a child's vaccination history.

The skin tattoo system including a microneedle patch and optionally an imaging device does not involve an invasive procedure. It is generally applied with a low requirement of medical skills or medical resources. It can be applied at clinic, school, farm or in the field.

The microneedle patch is not reused, avoiding cross-contamination. The needles dissolve a first application to the skin, leaving no microneedles or dyes for any subsequent use.

A. Applying: Self or Medical Professional

The patch is pressed upon the skin for five minutes dye pigment would be deposited sub-dermally upon the dissolution of the microneedles.

B. Data Storing, Transfer, and Reading

Generally, medical information is readily available by imaging the skin tattoo to access the impregnated information, and does not require a patient database. Alternatively, patient information including his/her medical history is stored and downloadable from a database with data collected and interpreted from the tattoo markings on patient.

### EXAMPLES

### Example 1. Photostability of Fluorophore Dyes: a Lanthanide Based Inorganic Dye, a Copper-Based Quantum Dot, and a Silver-Based Quantum Dot

Methods

Preparation of Dyes and Encapsulation in Microparticles

A lanthanide based inorganic dye material, IRDC3, was obtained. A copper-based quantum dot (copper QD) was synthesized containing a core-shell structure where the core contains copper-indium-selenide and a shell contains a zinc sulfide coating/film/overlay doped with aluminum, denoted as CuInSe2/ZnS:Al. The quantum yield of this copper-based quantum dot was between 40% and 50%. It was shown to be 7,000 times less toxic than CdTe QDs in vitro, and was used safely at 258 μg/kg in mice (target 3.36 μg/human) (Ding K, et al., Biomaterials 2014; 35:1608-17).

A silver-based quantum dot (silver QD) was synthesized containing a core-shell structure where the core contains silver-indium-selenide and a shell contains a zinc sulfide film doped with aluminum, denoted as AgInSe₂/ZnS:Al (Silver QD). The quantum yield of this silver-based quantum dot was up to 50%.

Results

These QDs were confirmed having a nanosized dimension under transmission electron microscopy (TEM). IRDC3 was examined under scanning electron microscopy (SEM).

Poly(methyl methacrylate) (PMMA) microparticles were prepared to encapsulate these fluorophores, resulting in encapsulated silver QD in PMMA particles at a loading of 60%; encapsulated copper QD in PMMA particles at a loading of 60%; and IRDC3 in PMMA particles at a loading of 1%.

1. Emission wavelengths did not overlap with melanin absorbance wavelengths.

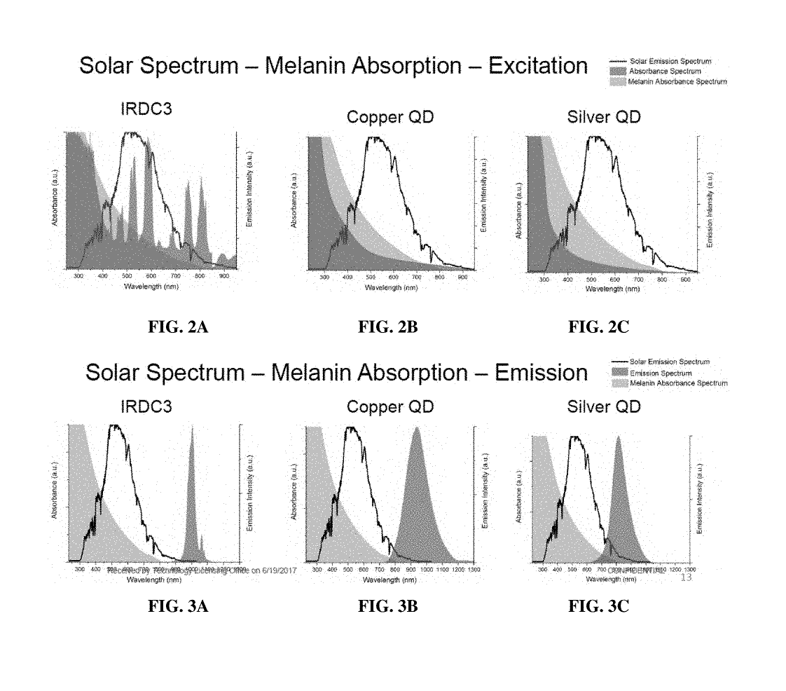

FIGS. 2A-2D show the absorbance spectra of IRDC3, copper QD, and silver QD, respectively. The absorbance spectrum of melanin is also shown in each spectrum.

FIGS. 3A-3C show the emission spectra of IRDC3, copper QD, and silver QD, respectively. The absorbance spectrum of melanin is also shown in each spectrum. The emission spectra of IRDC3, copper QD, and silver QD have little to no overlap with the absorbance spectrum of melanin, indicating that these three dyes were appropriate dye materials for delivery into the skin because their signals would not be absorbed by melanin, therefore detectable.

2. IRDC3 showed superior in vitro photostability to QDs.

Methods

Fluorophore suspensions were dropcast on slides. Samples were exposed to light simulating the solar spectrum at 7× intensity and imaged longitudinally over a simulated 84 days to observe photobleaching. Imaging was performed with 500 mW 808 nm laser expanded 15x×, band-pass 850-1100 nm emission filter, and a near-infra red camera.

Results

Dropcast IRDC3 intensity did not decrease during the simulated 84-day exposure period. Dropcast QDs performed poorly, likely due their broad excitation spectrum.

TABLE 1
Fluorescence intensity after 84-day photobleaching

| NIR Pigment | Remaining Fluorescent Intensity (%) |
|---|---|
| IRDC3 | 100.1 ± 2.2 |
| IRDC3 in PMMA | 80.7 ± 7.6 |
| Ag QD in PMMA | 15.3 ± 1.5 |
| Cu QD in PMMA | 6.9 ± 4.5 |

3. Copper QD showed superior ex vivo photostability to silver QD or IRDC3.

Methods

Fluorophores were tattooed into pigmented human abdominal skin obtained from a cadaver and imaged longitudinally. The signal from IRDC3 encapsulated in PMMA was so low that it had to be imaged separately from the other samples.

The initial intensities (normalized) before sun exposure of unencapsulated IRDC3, IRDC3 encapsulated in PMMA particles, copper QD encapsulated in PMMA particles, and silver QD in PMMA particles were 1.00±0.00, 0.12±0.01, 3.82±0.00, and 0.70±0.02, respectively.

Results

Copper QDs were the brightest at both the beginning and the end of the 84-day simulated sun exposure.

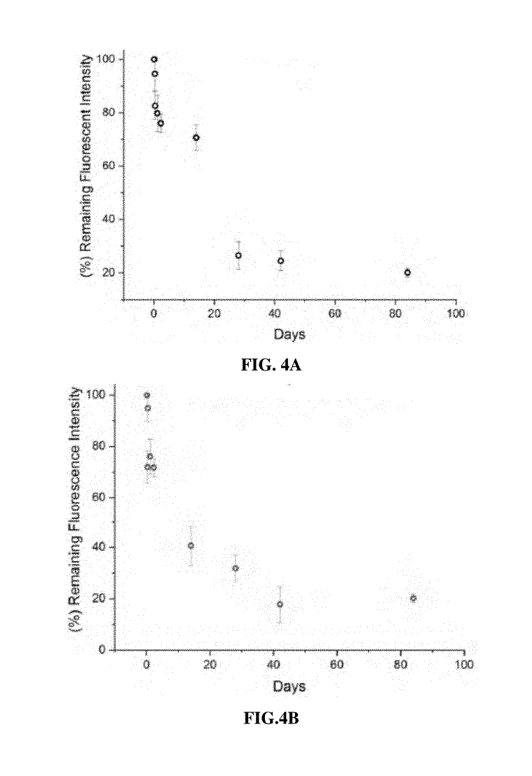

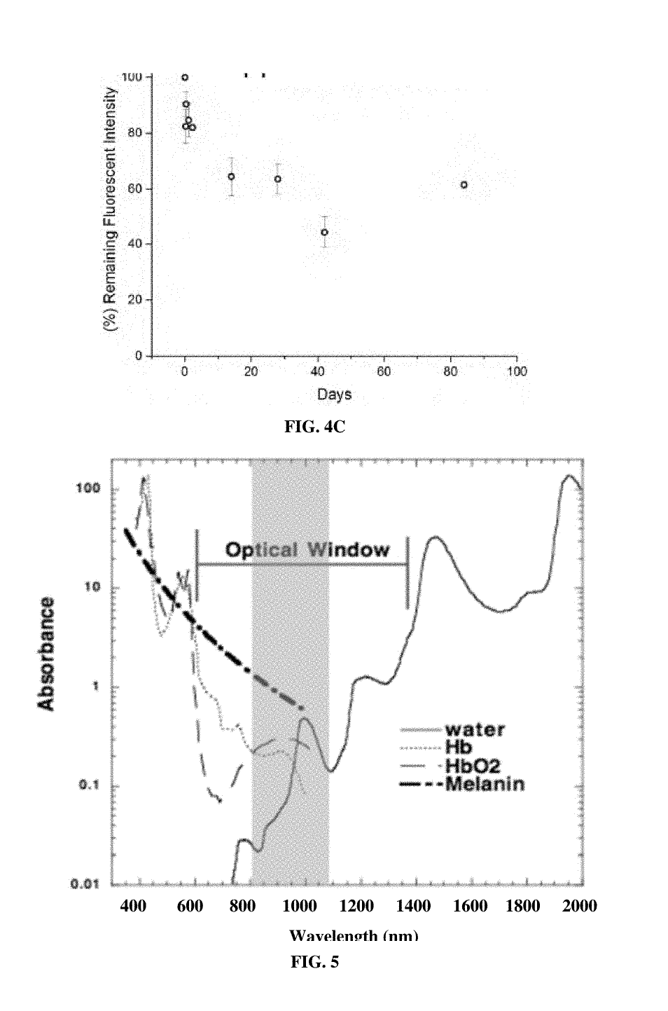

FIGS. 4A-4C show the ex vivo photostability of IRDC3, silver QD in PMMA, and copper QD in PMMA, respectively, over the course of the study. Table 2 shows the remaining fluorescent intensity (%) at the end of the study.

TABLE 2
Ex vivo fluorophore photostability after
three months of simulated exposure.

| Fluorophore | Remaining Fluorescent Intensity (%) |
|---|---|
| IRDC3 | 20.0 ± 4.5 |
| IRDC3 in PMMA | 3.4 ± 0.1 |
| Au QD in PMMA | 20.1 ± 1.7 |
| Cu QD in PMMA | 61.6 ± 1.3 |

4. Photostability in human cadaver skin.

Table 3 summarizes the percentage of remaining signals of dyes in human cadaver skin after 3-month simulated exposure.

TABLE 3
Comparison of the remaining signals (%) of each dye following
3-mon simulated exposure between dropcast on quartz slide
and tattooed under human cadaver skin.

| | Dropcast on | Tattooed Under |
|---|---|---|
| NIR Pigment | Quartz Slide | Human Cadaver Skin |
| IRDC3 | 100.1 ± 2.2 | 20.0 ± 4.5 |
| IRDC3 in PMMA | 80.7 ± 7.6 | 3.4 ± 0.1* |
| Ag QD in PMMA | 15.3 ± 1.5 | 20.1 ± 1.7 |
| Cu QD in PMMA | 6.9 ± 4.5 | 61.6 ± 1.3 |

IRDC3 experienced a greater loss of intensity when under pigmented skin than when directly exposed to light.

Copper QD performed much better under pigmented human skin than when directly exposed to light, probably because melanin helped absorb UV and visible light, as shown in FIG. 2B. FIG. 5 is a spectra of absorbance over wavelength (nm) for water, Hb, HbO₂, and melanin.

### Example 2. Evaluation of Lanthanide Based Inorganic Dyes IRDC2, IRDC3, IRDC4, IRDC5, and IRDC6

A custom-built system with a complementary metal oxide semiconductor (CMOS) camera was used to image efficiently in the near infra red (NIR) range. The system contained a laser source, a beam expander, and a mirror in this sequence on a similar horizontal level, such that the focused laser was reflected at the mirror to land a spot on a table where samples were located. The system was compatible for imaging NIR dyes with an emission wavelength in the range of 800-1100 nm. Dyes with the highest signal-to-noise ratios were selected using this system.

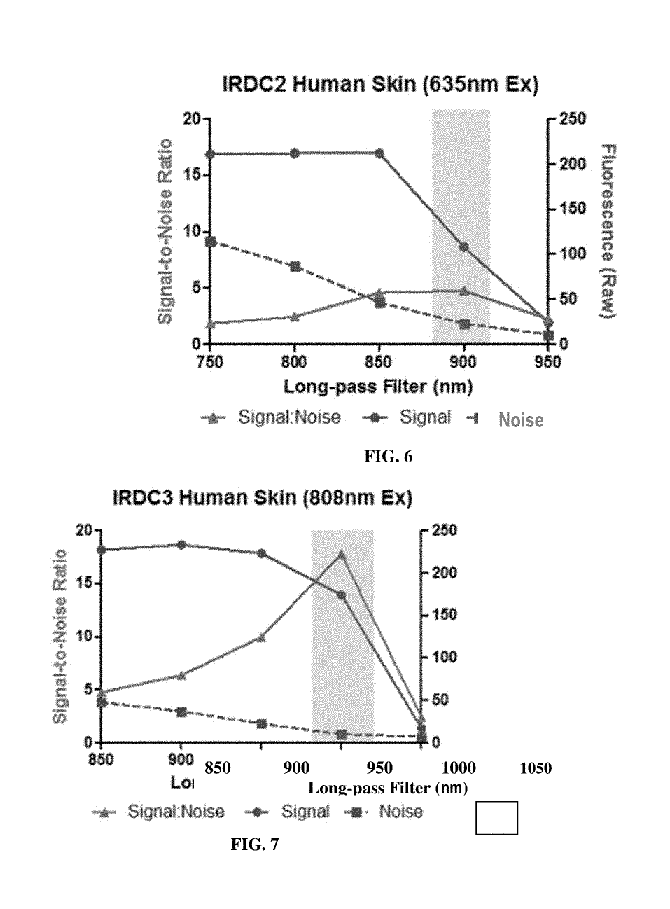

A lanthanide-based NIR dye, IRDC2, had an excitation wavelength below 700 nm and a sharp emission peak at 880 nm and 1070 nm. The quantum yield of it was approximately 85%. FIG. 6 shows under an excitation wavelength of 635 nm imaging IRDC2 through pigmented human skin, different emission wavelengths resulted in different signal-to-noise (S/N) ratios: for 700 nm, S/N=1.87; for 750 nm, S/N=1.84; for 800 nm, S/N=2.44; for 850 nm, S/N=4.58; for 900 nm, S/N=4.75; for 950 nm, S/N=2.24. Therefore, the optimal S/N (4.75) for IRDC2 was achieved at 635/900 nm in pigmented human skin when imaged in ambient light.

Another lanthanide-based NIR dye, IRDC3, had highest peaks of excitation around 800-830 nm and emission around 970-1030 nm. Its quantum yield was approximately 65%. FIG. 7 shows signal-to-noise ratio of IRDC3 in human skin for emission at different wavelengths as allowed through different long-pass filters (LPFs) when excited at 808 nm using a laser diode: for LPF=850 nm, S/N=4.75; for LPF=900 nm, S/N=6.34; for LPF=950 nm, S/N=9.95; for LPF=1000 nm, S/N=17.76; for LPF=1050 nm, S/N=2.40. When images were collected with integrated smartphone in normal ambient light, individual dots in an array were detected both in pig skin and pig skin covered in pigmented chicken skin.

When imaged with a 900 nm long-pass filter, IRDC3 in human skin had different S/N when excited at different wavelengths: for 635 nm, S/N=3.12; for 670 nm, S/S=2.56; for 780 nm, S/N=9.16; for 808 nm, S/N=6.34; for 830 nm, S/N=4.76; for 850 nm, S/N=2.47.

Another lanthanide-based NIR dye, IRDC4 had a red excitation and an NIR emission. It had a very low S/N in human skin even with optimal laser and LPF. When excited at 635 nm: for LPF=700 nm, S/N=1.88; for LPF=750 nm, S/N=1.74; for LPF=800 nm, S/N=2.08; and for LPF=850 nm, S/N=1.95.

Another lanthanide-based NIR dye, IRDC5 had a red excitation and an NIR emission. It also had a very low S/N in human skin even with optimal laser and long-pass filter. It had better S/N than IRDC4 due to the emission shift. When excited at 635 nm, IRDC5 in human skin emitted wavelengths that had different S/N ratios when different filters were used: for 700 nm filter, S/N=3.63; for 750 nm filter, S/N=1.99; for 800 nm filter, S/N=2.83; and for 850 nm filter, S/N=2.68.

Of the above assayed lanthanide dyes, IRDC3 and IRDC2 were promising candidates. Their optimal S/N was at high wavelengths, which helped reduce both pre-excitation and post-emission light absorption by melanin and tissue. The signal-to-noise ratio may be improved using a higher laser power (e.g., from 0.05 mW/mm² increased to 10 or 100 mW/mm²) or filters as discussed above. Here <0.07 mW/mm² was used, whereas generally a laser pointer is between 6 and 127 mW/mm². Increased laser power generally does not damage the skin. The signal-to-noise ratio may also be improved by using band-pass filters and/or removing ambient light during imaging.

### Example 3. Evaluation of the Effects of Size and Sharpness on Pain Associated with Applying the Microneedle to Skin and Dissolution in Skin

Administering the imaging agents in polymeric particles which are incorporated into polymeric microneedles increases reproducibility, sensitivity and ease of manufacturing.

Other advantages of this include low cost, ease of disposal (drop into bucket of bleach), and ability to deliver larger materials, thereby increasing the contrast to surface-adsorption ratio.

Studies were conducted to optimize the microneedle diameter, length, shape, and incorporation of particles.

Materials and Methods

Microneedles composed of 78% polyvinyl alcohol (PVA) and 22% polyvinylpyrrolidone (PVP) were produced using a micromolding technique. Dyes were facilely loaded by blending and casting into microneedle molds. Conical shaped (or pencil shaped) microneedles were mechanically stable.

Microneedle loaded with 20% IRDC3 was prepared for clear depiction of the dimension of the microneedle and the loading of a dye. This microneedle has a near cylindrical body of a length of 1.25 mm, a diameter of close to 0.3 mm, and a conical tip of 0.25 mm long. Under imaging, the dye was present not only at the tip but also a substantial portion of the body due to the overloading for depiction purpose.

Microneedle of a similar dimension but loaded with 17% silver QDs in PMMA particles was also prepared and imaged. A 4×4 array of microneedles, each of a similar dimension, loaded with 17% copper QDs in PMMA particles was prepared on a PDMS patch. The microneedles in the 4×4 array were spaced such that the array was 1 cm×1 cm.

Microneedles were fabricated to be 300 microns at their widest point and 1.5-2.0 mm long, which corresponded to 1.5 on a pain scale of 1-10.

Results

Microneedles dissolved to less than 50% of their initial heights within 5 minutes of skin application left behind a small puncture hole in the human abdomen skin that would close up immediately in living tissue.

Table 4 summarizes the dimensions of the microneedles and any associated pain to the subject and penetration forces.

TABLE 4
Dimensions, associated pain, and penetration
forces of microneedles.

| | Outer diameter | | Penetration Force (N) |
|---|---|---|---|
| Gauge | (microns) | Pain (%) [95% CI]* | [95%]* |
| 28 | 362 | 19.2 [14.2-24.1] | 0.32 [0.30-0.34] |
| 30 | 311 | 15.0 [10.1-20.0] | 0.29 [0.27-0.30] |
| 32 | 235 | 14.6 [9.6-19.6] | 0.25 [0.23-0.26] |

*Values reported in Praestmark K A, et al. BRM Open Diabetes Research and Care 2016; 4: e000266.

Masid MLS, et al. J Neurosci Nurs 47:E22-30 describes pain associated with needle diameter is minor and typically not statistically significant for needles of lengths of 4 mm, 6 mm, and 8 mm.

Needles only penetrate into the skin a distance of ½ to ⅔ of the needle height. A diameter of 300 microns, equivalent to a 30 G needles was selected. A longer length to reach non-shedding skin layer is required for long term marking. This equates to a length of about 1500 microns compared to 400 to 700 microns for most microneedles.

It is also important to optimize the shape and dimension to facilitate penetration so that it is as easy as possible, without the need for a separate applicator, minimizing signal to noise ratio, maintaining adherence until the tips of the microneedles which contain the imaging agent “break off” from the patch to remain in the skin, and as painless as possible.

As demonstrated below and in FIGS. 12A and 12B, an optimal shape is cone shaped. A cone shape is used as baseline (0). Since only the top portion of the microneedles needs to dissolve, increasing the ratio to 1:1 cylinder to cone, increases the volume four times. Increasing the ratio to 5:1 cylinder to cone increases the ratio to 9.3 times the volume.

These parameters minimize the penetration force while maximizing the payload. The result is that the optimal parameters are a height of 1500 microns and diameter of 300 microns. Modelling axial loading, bending, and buckling demonstrated that the optimal shape and dimensions were a 750 micron cone on top of a 750 micron cylinder.

With an applied force below 10 N to insert the microneedles, these parameters allow the use of an array of about 450 needles (range from 300 to 600, but higher resolution and stronger images obtained with more needles).

Ease of application is further enhanced by making the microneedles with a technique such as high-resolution 3D printing (2 photon) to produce very sharp tips.

### Example 4. Selection of Imaging Agent, Loading and Effect of Wavelength on Signal Attenuation

Organic fluorophores are bright but photobleach easily. Inorganic fluorophores are very photostable but exhibit low intensity, contain undesirable elements, and cannot be encapsulated easily using an emulsion process.

Improved signals were obtained by:

Increasing the loading of imaging agent in the microneedle tip.

Increasing particle size was increased to avoid macrophage clearance.

The imaging agent was also loaded preferentially into the microneedle tip to maximize signal retained in the skin.

The hardware was also optimized to increase the active imaging and decrease background signal.

Increased Loading of Polymeric Particles

Semiconductor nanocrystals (SNCs) are bright and photostable and can be made of biocompatible elements, although there are toxicity concerns due the presence of elements such as cadmium and lead. SNCs can also be modified to be soluble in organics to yield high percentage encapsulation (example 60% of total mass, using an oil-in-water emulsion).

Copper and silver based quantum dots with NIR emission at gram scale were synthesized and encapsulated in poly(methyl methacrylate) at 60% w/w using an emulsion process. Size was selected to minimize macrophage clearance. No observable adverse effects of the particles in vivo were observed over a period of two months.

FIG. 5 shows a schematic depicting the potential reduction of quantum yields of dyes due to absorbance of wavelengths by melanin and/or deeper tissue. When an excitation light shines on the skin, it may be absorbed by melanin and/or the deeper tissue before reaching the fluorophore. The excited fluorophore emits at a wavelength that may be absorbed by the tissue and/or melanin before emitting off the skin.

Loading more SNCs in polymeric particles (the exemplary polymer is a polymethylmethacrylate, PMMA) increased the signal per particle. An increase from 37.5% loading to 60% loading by weight was demonstrated using the emulsion process. This increased the signal by 60% (1.6×).

The following studies were conducted to maximize imaging.

Modified Cell Phone to Image:

For pigmented human skin, existing IVIS (in vivo imaging system) was ineffective due to filter limitations and light absorption by melanin. For example, a lanthanide based dye IRDC2 had strong signal attenuation through pigmented human skin when imaged with IVIS.

A modified cellular phone was able to image IRDC3 dye through human skin when excited and emitted at 808 nm and 950 nm, respectively.

Dyes with an excitation and/or emission wavelength in the UV range were not chosen for further studies due to one or more of the following reasons: may be visible under black light, have high background noise, and excitation and emission light are absorbed by melanin and tissue.

The signal-to-noise ratio for lower wavelengths was reduced from 50-150 down to <1.25. Melanin decreased tissue autofluorescence about 20-fold.

Compact Fluorescent (CFL) Bulb to Image:

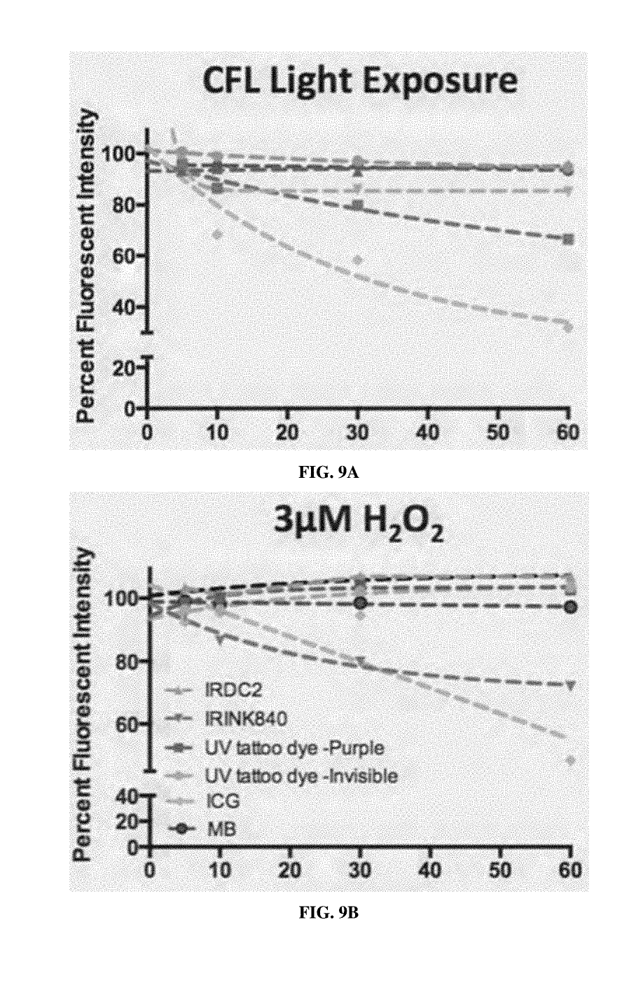

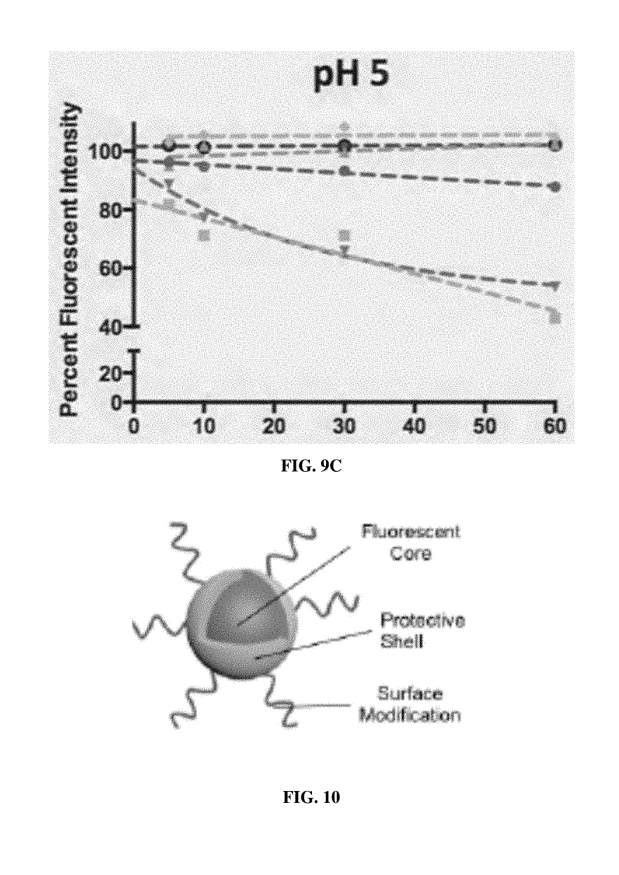

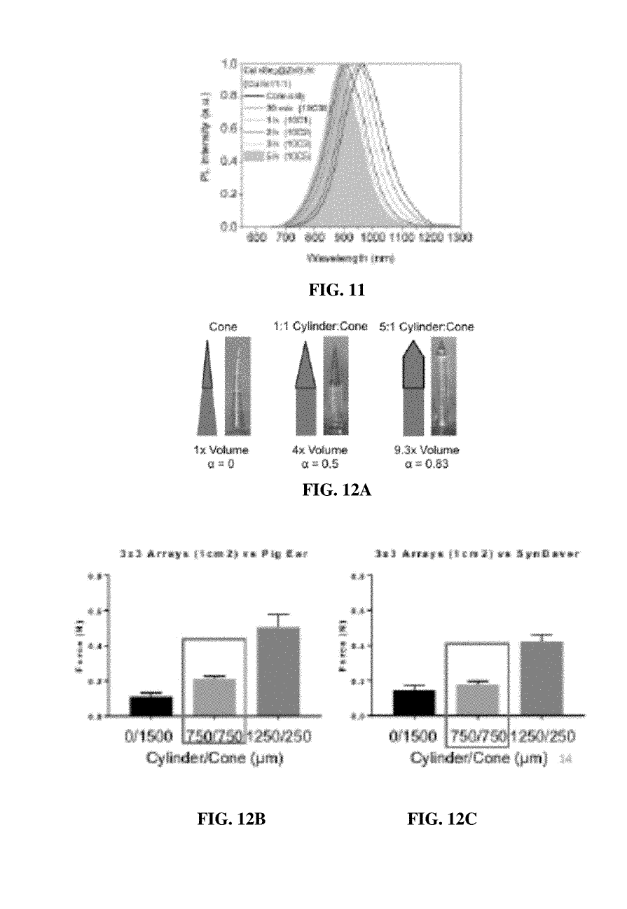

Dyes were prepared in solution (1 mM) or suspension (1 mg/mL) and exposed to light from a compact fluorescent (CFL) bulb (FIG. 9A). 55 fluorophores were tested including organic, encapsulated organic, inorganic, inorganic nanoparticle, tattoo, and semiconducting polymer dots.

Additional evaluations on the dyes to withstand oxidative stress were tested by submerging the dye in 3 mM or 3 micromolar hydrogen peroxide, and compared for signal before and after such treatment (FIG. 9B). 23 fluorophores were tested.

The pH stability of the dyes was evaluated by submerging in different pH ranging from 1 to 13 (FIG. 9C). 17 fluorophores were tested.

The tested dyes were in different categories and were summarized in Table 5.

TABLE 5
Different types of dyes for inclusion in microneedles.

| Dye Category | Emission | Performance |
|---|---|---|
| Lanthanide based | NIR (600-1100 nm) | Mostly chemically |
| inorganic dyes | | stable |
| (IRDC2-6) | | Good photostability |
| | | seen in solution |
| Commercial UV tattoo | UV (200-300 nm) | Chemically stable |
| dyes (encapsulated in | | Good photostability |
| toluenesulfonamide | | seen in solution in |
| resin) | | vitro |
| Xanthene Dyes | Visible (520-550 nm) | Poor photostability |
| (Fluorescein, | | Undesirable wavelength |
| Rhodamine, | | |
| Alexa Fluor) | | |
| Cyanine dyes | NIR (600-800 nm) | Poor chemical stability |
| (CY5, CY7, IR780) | | Poor photostability |
| Boron-dipyrromethene | Visible (450-500 nm) | Chemically stable |
| dyes | NIR 600-650 nm) | Average photostability |
| | | Undesirable wavelengths |

An example of a UV dye is INVISIBLE YELLOW which excites at 365 nm and emits at 549 nm. When imaged under ambient indoor light, the dye was visible by camera and naked eye when there was a high dye loading in microneedles.

Accelerated Photobleaching Setting:

Accelerated photobleaching was achieved with the SOLAR LIGHT 16S-300-006, which mimics the light spectrum of the sun. This xenon lamp-based unit simulated solar irradiance at sea level up to a factor of 7 sun equivalents, allowing for quick simulation of long-term degradation. It would be applicable in reliably testing photobleaching equivalent to five years or greater. In using SOLAR LIGHT, a cooling stage kept a sample at 37° C. and a passive flow tube counteracted evaporation. This unit also abides to the American Section of the International Association for Testing Materials (ASTM), the European Cosmetic and Perfumery Association (COLIPA), the International Organization for Standardization (ISO), and the U.S. Food and Drug (FDA) regulations in laboratory standards for photo-degradation testing.

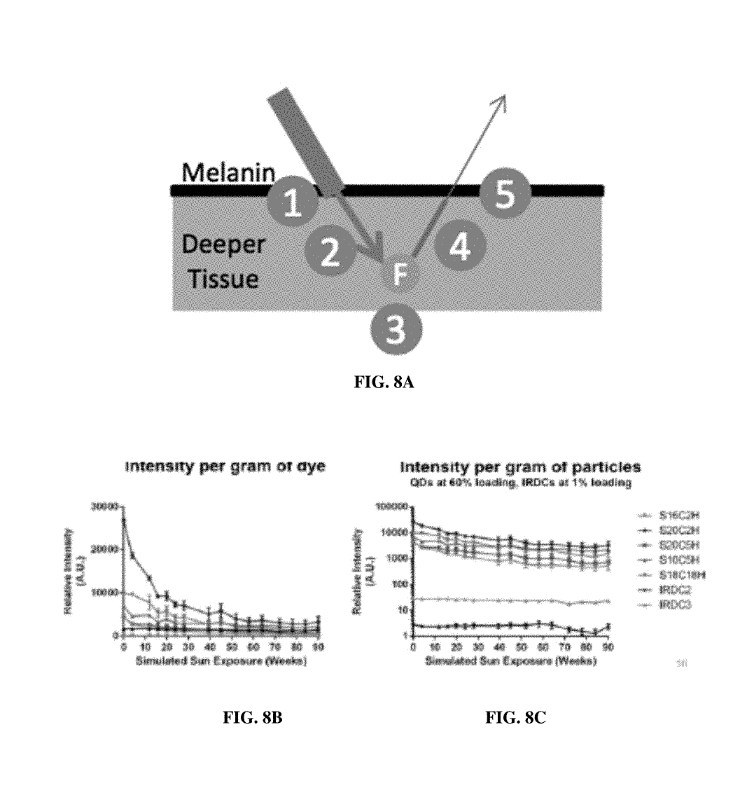

FIGS. 8A, 8B and 8C showed that the signal was lost early, problem due as a result of the defect-heavy proportion of SNCs bleaching easily. This emphasizes the importance of few defects. FIG. 8B shows that IRDC2, an inorganic heavy metal-containing dye, overtakes some of the quantum dotsat long time points because it is very stable. FIG. 8C shows that it is important to select the optimal method for encapsulation, with inorganic dyes not encapsulating efficiently with a solid/oil/water emulsion.

### Example 5: Effect of Particle Size and Location in Microneedle

Previous studies had SNCs distributed throughout the needles, leading to few microparticles at the tip where the needle dissolves and releases into the skin.

This was changed using a two-step process to increase the imaging agent particles in the tip of the microparticles.

Larger microparticles that were less likely to be phagocytized, i.e., greater than 14 micron, up to 30 micron, most preferably about 20 to 25 microns.

In the new process, the microparticles are suspended in water, dried, then back-filled in the microneedle solution, resulting in all of the microparticles being in the deliverable microneedle tip.

### Example 6: Toxicity Testing

The particles were tested to insure lack of toxicity.

Approximately 1000× the microneedle delivered dose was injected subcutaneously into mice.

The particles remained at the injection site. No clinical signs of morbidity were observed over a two month period of time.

Modifications and variations of the present invention will be apparent to those skilled in the art from the foregoing detailed description and are intended to come within the scope of the following claims. Individual references cited above are specifically incorporated by reference to the extent required.

We claim:

1. A microneedle array structure comprising
a flexible base element and
a plurality of biodegradable microneedles each having a first end and a second sharpened end for penetration of skin,
the microneedles extending outwardly from the base element at the first end of the microneedles,
The microneedles comprising therapeutic, prophylactic and/or diagnostic agent and/or dye,
wherein the microneedles are released from the base element within 15 minutes of administration into the skin.

2. The microneedle array structure of claim 1 wherein the therapeutic, prophylactic or diagnostic agent and/or dye is microencapsulated prior to incorporation into the microneedles.

3. The microneedle array structure of claim 1 wherein the microneedles are formed of biodegradable polymer or a sugar composition.

4. The microneedle array structure of claim 1 wherein the dye is selected from the group consisting of inorganic nanocrystals, lanthanide-based dyes, other fluorophores, and non-fluorescent imaging agents.

5. The microneedle array structure of claim 1 wherein the dye is carbon or a tattoo ink, or a cosmetic ink.

6. The microneedle array structure of any of claim 1 wherein the dye is a near infrared imaging agent with an excitation wavelength and an emission wavelength in the near infrared range.

7. The microneedle array structure of claim 6 wherein the dye is selected from the group of inorganic nanocrystals selected from copper-based quantum dots or silver-based quantum dots.

8. The microneedle array structure of claim 1 wherein the microneedles contain dye and form a pattern for identification of the individual, medical treatment, date, location, or combination thereof.

9. The microneedle array structure of claim 1 wherein the microneedles contain therapeutic, prophylactic or diagnostic agent.

10. The microneedle array structure of claim 9 wherein the agent is a vaccine.

11. The microneedle array structure of claim 1 comprising dye not visible in visible light but visualized in infrared light, ultraviolet light or by fluoroscopy.

12. The microneedle array structure of claim 1 wherein the arrays are sequentially numbered.

13. The microneedle array structure of claim 1 in a kit comprising an imaging device comprising a source for emitting a wavelength and optionally an optical filter for detection.

14. The microneedle array structure of claim 1 wherein the agents to be delivered are preferentially located in the tip of the microneedle which remains in the body after the needle dissolves sufficiently for the flexible base to fall off.

15. The microneedle array structure of claim 1 wherein the microneedles comprise a conical structure, preferably being a combination of conical and cylindrical structures.

16. A method of providing identification and/or tattooing and/or delivery of a therapeutic, prophylactic or diagnostic agent comprising applying to the skin of an individual the microneedle array structure of claim 1.

17. The method of claim 16 wherein the individual is an animal.

18. The method of claim 16 wherein the microneedle array structure administers a vaccine and identifies the vaccine and date and/or geographic location of the vaccination.

19. The method of claim 16 wherein the individual is in need of cosmetic tattooing.

20. The method of claim 16 wherein the individual is a military person.
